(19) United States
(12) Patent Application Publication
(10) Pub. No.: US 2017/0319730 A1
(43) Pub. Date: Nov. 9, 2017

(54) FRESHENING PRODUCT COMPRISING AN AQUEOUS PERFUME COMPOSITION CONTAINED IN A PRESSURIZED PLASTIC CONTAINER

(71) Applicant: The Procter & Gamble Company, Cincinnati, OH (US)

(72) Inventors: Chisomaga Ugochi NWACHUKWU, Cincinnati, OH (US); John Andrew MCDANIEL, Midd;etown, OH (US); Zaiyou LIU, West Chester, OH (US); Andrew William FRANCKHAUSER, Batavia, OH (US)

(21) Appl. No.: 15/661,205

(22) Filed: Jul. 27, 2017

Related U.S. Application Data
(63) Continuation of application No. 14/283,679, filed on May 21, 2014.

(51) Int. Cl. A61L 9/012; B65D 83/14; B65D 83/38

(52) U.S. Cl. CPC A61L 9/012; B65D 83/38; B65D 83/752

(57) ABSTRACT

Improved freshening products comprising an aqueous perfume formulation contained in a pressurized plastic container having a hoop tensile strain of about 0.1% to about 2% are provided.

### FIELD OF THE INVENTION

The present invention relates to improved freshening products comprising an aqueous perfume composition contained in a pressurized plastic container and methods thereof.

### BACKGROUND OF THE INVENTION

Pressurized containers for dispensing freshening compositions are known in the art and are typically constructed of metal in order to withstand the internal pressure of aerosols. Pressurized plastic containers containing a freshening composition may be desirable for cost, aesthetic (e.g. clear container), and recyclability advantages. It has been found, however, that the interaction of the perfume mixtures present in freshening compositions with plastic containers, especially when under pressure, may result in a phenomenon called crazing. Crazing is the appearance of small cleaves in the plastic, resembling cracks. Crazing is believed to be affected by the morphology of the plastic container, the strain on the container, and/or the chemistry of the composition contained in the container. Crazing is undesirable from both an aesthetic and functional point of view.

Manufacturers have attempted many techniques to minimize/avoid crazing in pressurized plastic containers due to certain chemical interactions of the formulations contained therein. These techniques include adjusting geometries and thicknesses of the container wall, applying a coating layer to the container wall, crystallizing certain portions of the container, and adjusting certain formulation chemistries. Attempts to overcome crazing problems are shown, for example, in U.S. Pat. No. 7,303,087 and WO 2011/088093. However, these approaches have not proven entirely satisfactory where a perfume mixture may be the primary active in the formulation (e.g. air freshening sprays). Further, some of the previous approaches require added production steps and/or costs (e.g. crystallizing neck portions, adding coatings, and constructing thicker walls than required for safety) and may be viewed as environmentally unfriendly.

As such, there remains a need for improved freshening products comprising a pressurized plastic container containing an aqueous perfume composition having a perfume mixture and an aqueous carrier that minimizes crazing.

### SUMMARY OF THE INVENTION

The invention comprises a freshening product comprising an aqueous composition comprising a perfume mixture; and a pressurized plastic container containing said aqueous composition, wherein said container comprises a hoop tensile strain of about 0.1% to about 2%.

In another embodiment, the present invention includes an air freshening composition comprising an aqueous composition comprising a perfume mixture, a solubilizer, and greater than 70%, by weight of said composition, of an aqueous carrier; and a pressurized plastic container containing said aqueous composition, wherein said container comprises a hoop tensile strain of about 0.1 to about 1% and wherein said container comprises a gage pressure greater than about 414 kPa.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a fragmentary, frontal view of a container, according to the present invention, having a flange and lower neck region which is usable to calculate hoop tensile strain according to the present invention;

FIG. 2 is a fragmentary, vertical sectional view taken along lines 2-2 of FIG. 1;

FIG. 3 is an enlarged view of the neck region denoted with the boundary region 3 shown in FIG. 2;

FIG. 4 fragmentary vertical sectional view of a plastic container having an optional flange, and showing the respective axial midpoint MP of the neck of such plastic container.

### DETAILED DESCRIPTION OF THE INVENTION

The present invention provides a freshening product comprising a pressurized plastic container having a specified hoop tensile strain and an aqueous perfume composition comprising a perfume mixture and an aqueous carrier. This combination has shown to minimize/prevent crazing in plastic containers.

### Aqueous Composition

The aqueous composition of the present invention comprises a perfume mixture and an aqueous carrier. The final pH of the aqueous composition herein may be from about 1 to about 11, alternatively from about 3 to about 10, alternatively from about 4 to about 8.

Perfume Mixture

The perfume mixture may comprise one or more of any known perfume material. “Perfume” refers to organic substances that are included in a product to provide a desired olfactory property, whether scented, low-scent/unscented. A perfume may include chemicals that are aromatic, aliphatic, ionone, hydrocarbon, alcohol, aldehyde, ketone, and ester.

Exemplary perfume materials are disclosed in U.S. Pat. Nos. 5,663,134; 5,670,475; 5,783,544; 5,939,060; and 6,146,621. In some embodiments, the aqueous composition is substantially free of or free of a flavorant. A flavorant is an edible chemical that is added to food and beverage products to alter the taste of the food or beverage product. Where a perfume mixture is free of a flavorant, the perfume mixture is free of flavorants including perfume materials that are known to be used as flavorants in the food and beverage industry. Having a perfume mixture that is free of flavorants can help provide improved hedonic benefits of the aqueous composition.

While any perfume material known to provide an olfactory benefit may be used in the present invention, suitable perfumes are listed in Table 1.

TABLE 1

| Perfume Material Name | CAS |
|---|---|
| 2 6-Nonadien-1-Al/10% In DPG | 17587-33-6 |
| Adoxal | 141-13-9 |
| Allyl Amyl Glycolate | 67634-00-8 |
| Alpha Damascone | 24720-09-0 |
| Benzyl Acetate | 140-11-4 |
| Benzyl Salicylate | 118-58-1 |
| Beta Gamma Hexenol | 928-96-1 |
| Bourgeonal | 18127-01-0 |
| Camphor Gum | 76-22-2 |
| Cedarleaf | 8007-20-3 |
| Cis 3 Hexenyl Acetate | 3681-71-8 |
| Cis-3-Hexenyl Salicylate | 65405-77-8 |
| Citral | 5392-40-5 |
| Citronellol | 106-22-9 |
| Cyclo Galbanate | 68901-15-5 |
| Cymal | 103-95-7 |
| Decyl Aldehyde | 112-31-2 |
| Dihydro Myrcenol | 18479-58-8 |
| Ethyl 2 Methyl Pentanoate | 39255-32-8 |
| Eucalyptol | 470-82-6 |
| Eugenol | 97-53-0 |
| Floralozone | 67634-15-5 |
| Florhydral | 125109-85-5 |
| Helional | 1205-17-0 |
| Hexyl Cinnamic Aldehyde | 101-86-0 |
| Hydroxycitronellal | 107-75-5 |
| Ionone Beta | 14901-07-6 |
| Iso E Super Or Wood | 54464-57-2 |
| Laevo Carvone | 6485-40-1 |
| Lauric Aldehyde | 112-54-9 |
| Lavandin Grosso | 78-70-6 |
| Lemon Cedrat | 5989-27-5 |
| Ligustral Or Triplal | 68039-49-6 |
| Lime Oil #6 | 5989-27-5 |
| Linalool | 78-70-6 |
| Linalyl Acetate | 115-95-7 |
| Litsea Cubeba | 5392-40-5 |
| Lyral | 31906-04-4 |
| Melonal | 106-72-9 |
| Methyl Benzoate | 93-58-3 |
| Methyl Dihydro Jasmonate | 24851-98-7 |
| Methyl Dioxolan | 6413-10-1 |
| Methyl Iso Butenyl Tetrahydro Pyran | 16409-43-1 |
| Methyl Phenyl Carbinyl Acetate | 93-92-5 |
| Nopyl Acetate | 128-51-8 |
| Octyl Aldehyde | 124-13-0 |
| Oxane | 59323-76-1 |
| P.T.Bucinal | 80-54-6 |
| Polysantol | 107898-54-4 |
| Rhubafuran | 82461-14-1 |
| Silvanone Ci | 3738-00-9 |
| Thymol Nf | 89-83-8 |
| Verdox | 88-41-5 |

### Aqueous Carrier

The aqueous composition of the present invention also comprises an aqueous carrier. The aqueous carrier which is used may be distilled, deionized, or tap water. Water may be present in any amount for the composition to be aqueous. In some embodiments, water may be present in an amount of about 70% to about 99.9%, or about 80% to about 99.9%, or about 85% to 99.9%, or about 90% to about 99.5%, or about 92% to about 99.5%, or about 95%, by weight of said aqueous composition.

Optional Ingredients

The aqueous composition may also have less than about 30%, or less than about 10%, or less than about 5%, by weight of the composition, of alcohol. However, the volatile low molecular weight monohydric alcohols such as ethanol and/or isopropanol should be limited since these volatile organic compounds may contribute both to flammability problems and environmental pollution problems. If small amounts of low molecular weight monohydric alcohols (e.g., ethanol, methanol, and isopropanol, or polyols, such as ethylene glycol and propylene glycol) are present in the composition of the present invention due to the addition of these alcohols to such things as perfumes and as stabilizers for some preservatives, the level of monohydric alcohol may be about 1% to about 5%, alternatively less than about 5%, by weight of the aqueous composition.

The aqueous perfume composition of the present invention may be formulated into an aerosol freshening composition comprising malodor counteractants, particulate controlling polymers, emulsifiers and solubilizing surfactants to solubilize any excess hydrophobic organic materials, particularly any perfume materials. Other emulsifiers, solvents, solubilizers and surfactants as described in U.S. Pat. No. 7,998,403 or in US 2012/0288448A1 can be used to enhance performance of the aqueous composition. A suitable solubilizing surfactant, is a no-foaming or low-foaming surfactant. In one embodiment, the aqueous composition contains ethoxylated hydrogenated castor oil. One suitable hydrogenated castor oil is Basophor™, available from BASF. The solubilizer to perfume ratio in aqueous perfume composition may be about 2:1 or greater than 2:1.

### Pressurized Plastic Container

The aqueous composition of the present invention is contained in a pressurized plastic container 10. “Plastic” refers to any synthetic or organic material that can be molded or shaped, generally when heated, and then hardened into a desired form including, but not limited to, polymer, resin, and cellulose derivative. The plastic may be polymeric and may be partially, substantially, or entirely comprised of polyester; polyethyleneterephthalate (“PET”); polyethylene napthalate, polyethylene furanoate, polyamide; nylon 6/6, nylon 66, nylon 11, polycarbonate; polyoxymethylene; polyacrylonitrile; polyolefin; polyethylene, polypropylene, fluoropolymer; poly(butylene succinate); virgin, recycled, and regrind versions of the other polymer materials; bio-based and petroleum-based versions of the other polymer materials; and mixtures thereof. In one embodiment, the pressurized plastic container 10 comprises multiple layers of other polymer materials. By polymeric it is meant that the component is formed of a material which is plastic, comprises polymers, and/or particularly polyolefin, polyester or nylons. Thus, the entire pressurized plastic container 10 or, specific components thereof, may be free of metal, allowing for exposure to microwave energy.

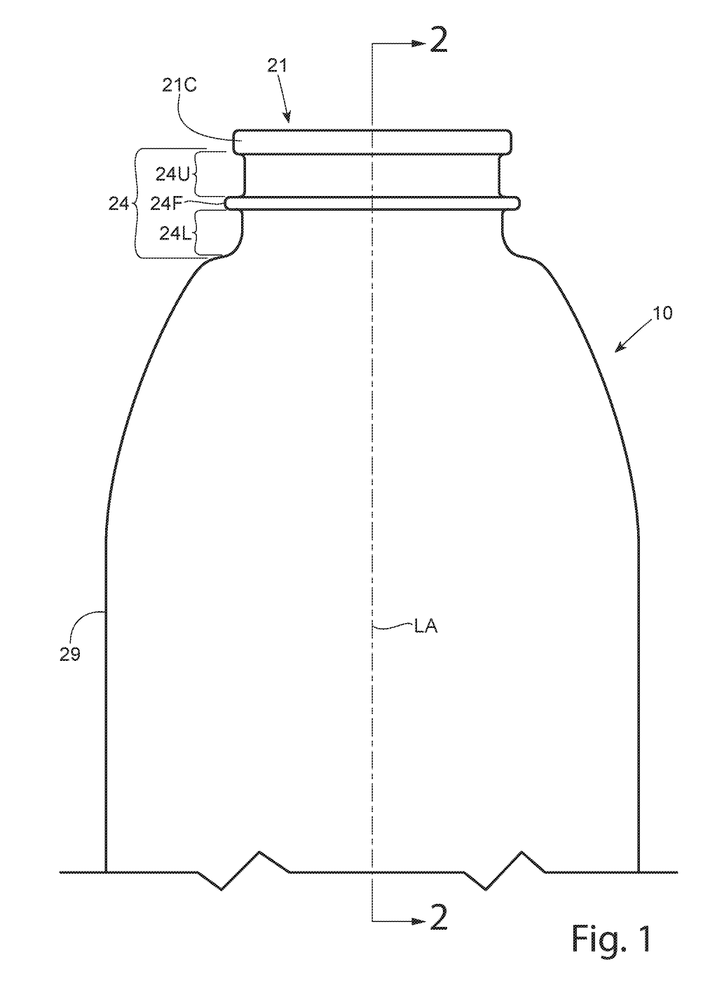

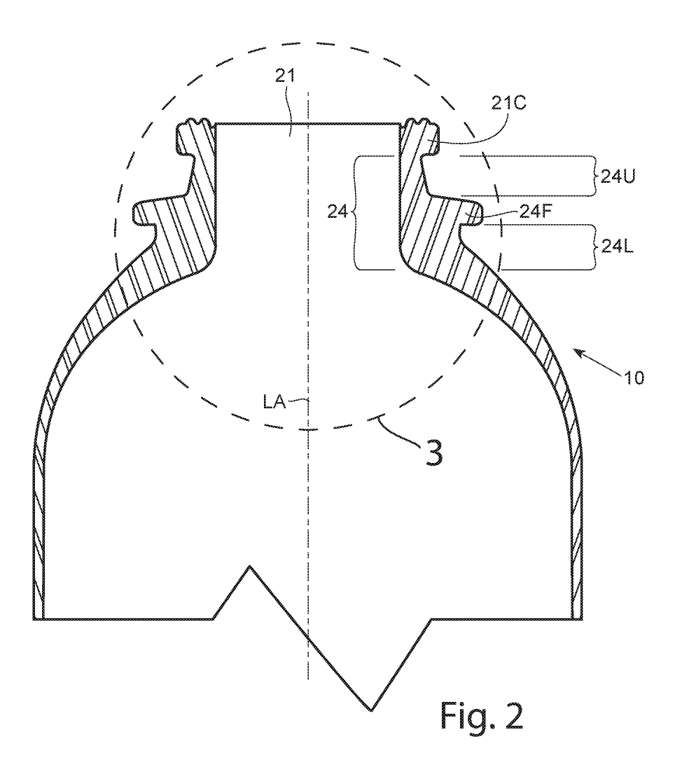

Referring FIGS. 1 and 2, as the top of the plastic container 10 is approached, the plastic container may have an opening 21. An optional crimp ring 21C may be circumjacent, and in a degenerate case circumscribe the opening 21. The optional crimp ring 21C may provide for attachment of an optional valve cup as is known in the art.

Below the optional crimp ring 21C, is a neck 24. The neck 24 may be of constant or variable cross section. The neck 24 may have an optional flange 24F. The optional flange 24F may extend radially outward from the neck 24, as shown and/or may extend radially inwardly. The flange 24F may circumscribe the neck 24 or may be interrupted at various positions around the circumference to be discontinuous. The plastic container 10 may have a step in the outer wall of the neck 24. Such a plastic container may be made according to U.S. Pat. No. 6,971,530. The flange 24F may be disposed near the axial center of the neck 24, as shown, or may be juxtaposed with the top or bottom of the neck 24. The flange 24F may divide the neck into an upper portion 24U and a lower portion 24L. The neck 24 may have a lesser thickness at the top portion 24U than at lower portion 24L, or vice versa, to provide a differential thickness.

The lower portion 24L of the neck 24 may be above and/or superjacent the shoulder 25 of the plastic container 10. The shoulder 25 may flare radially outwardly from the lower portion 24L in a first transition region. The shoulder 25 may connect to the container sidewall 29. The shoulder 25 may particularly be joined to the sidewall 29 by a radius or second transition region. The plastic container 10 sidewall also defines a diameter if a round cross section is selected for the body.

When at least partially filled, or filled at least with 58%, of an aqueous perfume composition, the plastic container 10 is pressurized to an internal gage pressure of about 345 kPa (55 psi) to about 1000 kPa, or from about 414 kPa to about 1000 kPa, or from about 690 kPa (100 psi) to about 1000 kPa, or about 827 kPa (120 psi) to about 1000 kPag, or 935 kPa (135 psi) to about 1000 kPa. The final gage pressure of the plastic container 10 is 0 when the propellant is fully discharged from the container. Where residual aqueous perfume composition remains, the final gage pressure may be from about 0 to about 120 kPa.

The pressurized plastic container 10 includes a propellant. Any suitable propellant may be used. The propellant may comprise a hydrocarbon as known in the art, nitrogen, carbon dioxide, liquefied gas or hydrofluoro olefin (“HFO”), compressed air, and mixtures thereof. Propellants listed in the U.S. Federal Register 49 C.F.R. §1.73.115, Class 2, Division 2.2 are considered acceptable. The propellant may particularly comprise a trans-1,3,3,3-tetrafluoroprop-1-ene, and optionally a CAS number 1645-83-6 gas. Such propellants provide the benefit that they are not flammable, although the invention is not limited to inflammable propellants. One such propellant is commercially available from Honeywell International of Morristown, N.J. under the trade name HFO-1234ze or GWP-6.

If desired, the propellant may be condensable. By “condensable”, it is meant that the propellant transforms from a gaseous state of matter to a liquid state of matter in the container and under the pressures encountered in use. Generally, the highest pressure occurs after the container is charged with product but before that first dispensing of that product by the user. A condensable propellant provides the benefit of a flatter depressurization curve as product is depleted during usage.

The aqueous perfume composition of the present invention is delivered from the pressurized plastic container 10 which includes delivery components including but not limited to a valve to control flow and to seal the composition within the pressurized plastic container, a button actuator and a nozzle for dispensing the composition to the environment.

In some embodiments, the aqueous perfume composition contacts the inner face of the plastic container 10 and is not contained in a plastic container by a bag-in-can. In other embodiments, the aqueous perfume composition may be contained in a bag-in-can plastic container. The pressurized plastic container may hold from about 20 grams to about 300 grams, or at least about 20 grams, or at least about 120 grams, or at least about 130 grams, or at least about 150 grams.

The container 10 of the present invention comprises a hoop tensile strain from about 0.1% to about 2%, or from about 0.1% to about 1%, or from about 0.2% to about 0.6%. “Hoop tensile strain” is the resultant material deformation as a function of the internally applied force exerted circumferentially (perpendicular to the major longitudinal axis LA on every particle in the cylinder wall of a plastic container and is represented as a % value derived from the hoop tensile strain test method set forth in this application.

Test Method to Determine % Hoop Tensile Strain—Measurement of Pressurized Plastic Aerosol Container Strain

Optical (photographic 2D), X-ray (Computed Tomography 3D), or equivalent imaging techniques may be used to quantify the level of strain at specific locations on a pressurized aerosol container. The % hoop tensile strain determination is made by imaging the region of interest with and without pressure. Relative dimensional changes are used to calculate % hoop tensile strain.

Sample Preparation

Procure a minimum of six unused pressurized plastic aerosol containers and condition them for 24 hours in the ambient environment (24° C.+/−3° C.) prior to imaging.

Imaging

Secure each conditioned plastic container by the upper neck 24U in a collet or equivalent clamping device to keep the container stable and to prevent contact with surrounding surfaces. Orient the containers, relative to the imaging device, such that at least three containers will be imaged, displaying their widest diameter of the neck 24 and at least three other containers will be imaged displaying their smallest diameter of the neck. The container temperature, when imaged, should be held constant across the pressurized and unpressurized conditions.

For each sample, optimize the resolution by only imaging the region of interest—the lower neck 24L and filling the field of view. Fix the imaging device field of view/focal plane. Place a scale that occupies the field of view in the focal plane and capture the image to set the image scale. The minimum resolution is 100 pixels/mm or 64,000 voxels/mm³. Place the plastic container in the field of view/focal plane and capture the image using appropriate imaging techniques to maximize contrast between the plastic container and the environment. Relieve the aerosol pressure without moving the plastic container or altering the position of the field of view or focal plane. Allow the unpressurized plastic container to equilibrate for a minimum of 1 hour so that the external pressure is equal to the internal pressure, while ensuring that there is no movement of the plastic container, the field of view, or the focal plane. Capture the unpressurized plastic container image under the same imaging conditions as the pressurized plastic container.

Image Analysis:

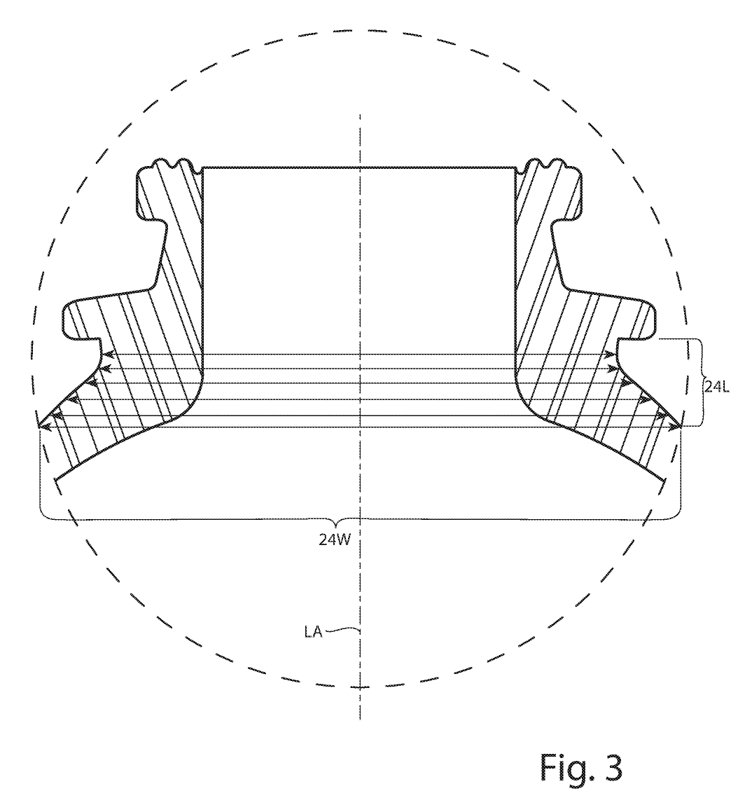

Using appropriate image analysis software, such as Image J (National Institutes of Health, Bethesda, Md., U.S.A.), or Geomagic Studio (Geomagic Inc., Morrisville, N.C., U.S.A.), set the dimensional scale in the images (pixels/mm or voxels/mm³) by using the previously captured scale image. Measure the outside diameter (“OD”) of the pressurized lower neck 24L image at 1 mm increments, defined as starting 1 mm below the flange 24F or below the neck 24 MidPoint MP and extending 5 mm vertically toward the bottom of the container 10, generating a total of 6 OD measurements as denoted with lines 24W shown in FIG. 3. Precisely identify the six locations where outside diameters were measured in the pressurized neck image, then remeasure the outside diameter at these same six locations in the unpressurized neck image. Convert the linear outside diameter dimensional change to % hoop tensile strain per the following equation, and report the maximum % hoop tensile strain value from the six locations measured on each container:

Hoop Tensile Strain at location x=(ODPₓ−ODUₓ)/ODPₓ*100
where ODPₓ=Outside diameter pressurized at location x, where x=1 to 6;
where ODUₓ=Outside diameter unpressurized at location x, where x=1 to 6; and
where the strain value recorded for each container is the maximum value obtained from the six locations measured.

The % Hoop Tensile Strain value reported is the maximum strain value recorded from the six (or more) individual containers imaged.

Plastic Container Lower Neck 24L Hoop Strain Determination Example:

Imaging

Image lower neck 24L, capturing the full width 24W while setting the focal plane at the maximum neck diameter. Image pressurized and post pressurization region as instructed in the imaging section above.

| | Post pressure | | | | | | | Strain | Strain | Strain | Strain | Strain | Strain | Max |
|---|---|---|---|---|---|---|---|---|---|---|---|---|---|---|
| Identification | time (minutes) | OD 1 | OD 2 | OD 3 | OD 4 | OD 5 | OD 6 | 1 (%) | 2 (%) | 3 (%) | 4 (%) | 5 (%) | 6 (%) | Strain (%) |
| Pressurized | | 23.86 | 21.16 | 20.48 | 20.14 | 19.84 | 19.73 | | | | | | | 0.30 |
| Unpressurized | 150 | 23.86 | 21.11 | 20.43 | 20.08 | 19.82 | 19.70 | 0.00 | 0.24 | 0.24 | 0.30 | 0.10 | 0.15 | |

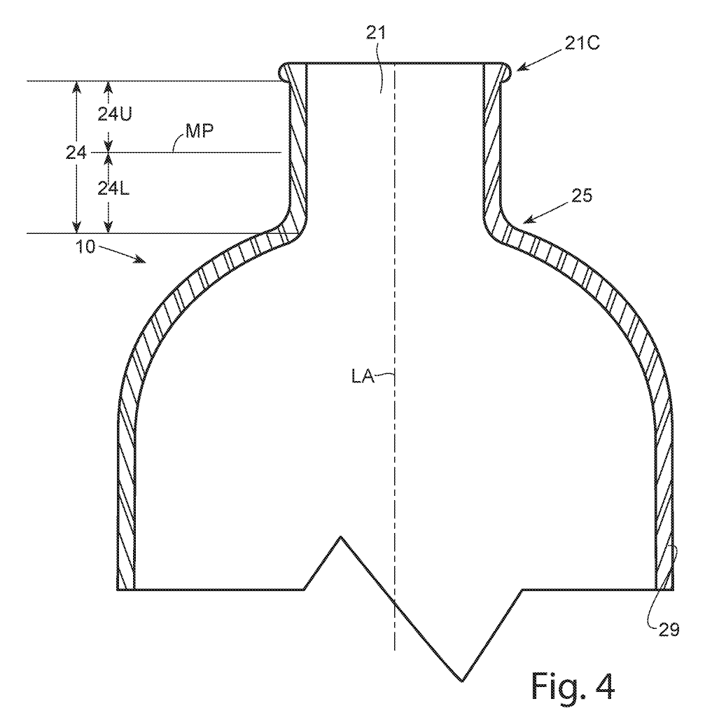

Referring to FIG. 4, the plastic container 10 may not have the optional flange 24F. In such case, the hoop tensile strain may be taken as a region below the axial midpoint MP of the neck 24. In determining the axial midpoint MP of the neck 24, only the portion of the neck 24 having constant cross section, if present, is considered. If the neck 24 has a variable cross section, the axial midpoint MP is that point of the neck 24 intermediate and midway between the underside of the optional crimp ring 21C and the start of the shoulder 25. If the plastic container 10 does not have a crimp ring 21C, the top of the neck 24 is utilized instead. The axial midpoint MP of the neck 24 may be used in addition to or in place of the flange 24F for purposes described and claimed herein.

The hoop tensile strain and aqueous perfume composition of the present invention provide an acceptable crazing profile in accordance with Table 2. Crazing of less than 1 mm may be considered consumer acceptable. Crazing of 1 mm or greater through a plastic PET is consumer unacceptable as such crazing may compromise the function of the container under normal use conditions (e.g. may result in container rupturing when force is applied to it, for example, container dropping).

TABLE 2
Crazing Acceptance Scale

| Acceptability | Description/Observation | Score |
|---|---|---|
| Target | No crazes/Undetectable | 100 |
| Acceptable | surface craze | 80 |
| Acceptable - Multiple | Multiple surface crazes | 65 |
| Manageable | Penetrating craze (<1 mm) | 50 |
| Manageable - Multiple | Multiple penetrating crazes (<1 mm) | 35 |
| Unacceptable | Substantial Penetrating craze (>1 mm) | 20 |
| Unacceptable - Multiple | Multiple Substantial | 0 |
| | penetrating crazes (>1 mm) | |

### Examples

### Example 1: Effect of Strain and Perfume Mixture on Crazing

Strain was introduced to injection molded PET test bars (127 mm long×12.7 mm wide×3.1 mm thick) by clamping to individual curved stainless steel fixtures, putting the exposed surface of the bars under bending strains of 0.5%, 1%, 2%, and 3%. A first set of three test bars were placed in direct contact with equal amounts of perfume mixture at each strain level for 72 hours at 23° C. Another set of three test bars were immersed in aqueous perfume composition shown in Table 3 at each strain level for 72 hours at 23° C.

TABLE 3

| | |
|---|---|
| Water | 92.4% |
| Alcohol | 5% |
| Perfume | 0.6% |
| Surfactants & Emulsifiers | 1.9% |
| Buffers & Other Stabilizers | 0.1% |

The test bars were removed from the fixtures, rinsed, dried then evaluated using a transmitted light stereomicroscope. The resulting crazing of the PET test bars was scored and averaged using the Crazing Acceptance Scale of Table 2.

Manageable to acceptable crazing scores resulted for PET bars under 0.5% to 2% strain and subjected to a perfume mixture in an aqueous composition. Results are reported in Table 4.

TABLE 4
Uncrazed Score

| | | | | |
|---|---|---|---|---|
| Tensile Strain | 0.5% | 1% | 2% | 3% |
| Perfume Mixture Only (i.e. No | 20 | 0 | 0 | 0 |
| Aqueous Carrier) | | | | |
| Perfume mixture + Aqueous Perfume | 50 | 50 | 35 | 0 |
| Composition of Table 3 | | | | |

### Example 2: Effect of Aqueous Perfume Composition with Other Ingredients on Crazing

Aqueous compositions in accordance with Table 5 were prepared.

TABLE 5

| | Composition A | Composition B |
|---|---|---|
| Water | 94.4% | 92.4% |
| Alcohol | 5% | 5% |
| Perfume | 0.6% | 0.6% |
| Surfactants & Emulsifiers | 0% | 1.9% |
| Buffers & Other Stabilizers | 0% | 0.1% |

Each aqueous perfume composition in Table 5 was packed into ten PET containers and pressurized. Each container contained about 1.8 g of perfume (which is about 0.6%, by total weight of the composition). A control set with 1.8 g perfume mixture only was also packed into ten PET containers and pressurized. All containers used for this experiment contained about equal amounts of perfume. All containers were under about 0.3% strain. The samples were stored at 50° C. for one month and then evaluated for crazing using the Crazing Acceptance Scale of Table 2. Each sample was repeated ten times and the corresponding uncrazed score was averaged across the ten containers tested per sample (n=10 for each sample).

Containers containing perfume in an aqueous Composition A or Composition B having surfactants, emulsifiers, buffers, and stabilizers improved the uncrazed score. Results are reported in Table 6.

TABLE 6
Uncrazed Score

| | |
|---|---|
| Perfume mixture only (no aqueous carrier) | 0 |
| Composition A | 70 |
| Composition B | 87 |

### Example 3: Impact of Pre-Treating Pressurized Plastic Containers with Aqueous Compositions

An aqueous composition in accordance with Table 7 was prepared.

TABLE 7

| | |
|---|---|
| Water | 93% |
| Alcohol | 5% |
| Surfactants & Emulsifiers | 1.9% |
| Buffers & Other Stabilizers | 0.1% |

Ten PET Containers were pre-treated with this aqueous composition by filling and storing them overnight (at least 12 hours) at constant room temperature (“Test Composition 1”). Another set of ten PET containers were similarly pre-treated with water by filling and storing them overnight at constant room temperature (“Test Composition 2”). A third set of ten PET containers were kept untreated and stored overnight at constant room temperature (“Test Composition 3”). The pre-treated PET containers were then emptied and allowed to dry again overnight. All PET containers were then exposed to equal amounts of perfume mixture (0.12 g), pressurized to 0.3% strain, and then stored at 50° C. for one month. Each plastic container was then evaluated for crazing and was graded according to the Acceptance Scale in Table 2.

Containers that were pre-treated with water resulted in less crazing than un-treated containers. Containers pre-treated with the aqueous composition of Table 7 performed better than both the pre-treated water container and the un-treated container. Results are reported in Table 8.

TABLE 8
Uncrazed Score for Pre-treated and Un-treated Plastic Containers

| | Uncrazed Score |
|---|---|
| Test Composition 1: perfume mixture only (no aqueous | 30 |
| carrier) in untreated container | |
| Test Composition 2: perfume mixture in containers pre- | 63 |
| treated with water | |
| Test Composition 3: perfume mixture in containers pre- | 98 |
| treated with the aqueous composition of Table 7 | |

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed:

1. A freshening product comprising:
an aqueous composition comprising a perfume mixture; and
a pressurized plastic container containing said aqueous composition, wherein said container comprises a hoop tensile strain of about 0.1% to about 2%.

2. The product of claim 1, wherein said hoop tensile strain is about 0.1 to about 1%.

3. The product of claim 1, wherein said hoop tensile strain is about 0.2 to about 0.6%.

4. The product of claim 1, wherein said container is under a gage pressure from about 345 kPa to about 1000 kPA.

5. The product of claim 1, wherein said container is under a gage pressure from about 827 kPa to about 1000 kPa.

6. The product of claim 1, wherein said container is pre-treated with water.

7. The product of claim 1, wherein said container is pre-treated with an aqueous composition comprising water and a solubilizer.

8. The product of claim 1, wherein said aqueous carrier is present in an amount greater than about 70%, by weight of said composition.

9. The product of claim 1, wherein said aqueous carrier is present in an amount greater than about 90%, by weight of said composition.

10. The product of claim 1, wherein said perfume mixture is present in an amount of about 0.01% to about 10%, by weight of said composition.

11. The product of claim 1, further comprising a propellant selected from the group consisting of: nitrogen, carbon dioxide, compressed air, hydrofluoro olefin, and mixtures thereof.

12. The product of claim 11, wherein said propellant is free of hydrocarbons.

13. The product of claim 1, further comprising a solubilizer selected from group consisting of: non-ionic surfactant, alcohol, glycol, and mixtures thereof.

14. The product of claim 1, wherein said composition comprises less than about 30%, by weight of said composition, of alcohol.

15. The product of claim 1, wherein said composition comprises less than about 5%, by weight of said composition, of alcohol

16. An air freshening product comprising:
an aqueous composition comprising:
a perfume mixture;
a solubilizer; and
greater than about 70%, by weight of said composition, of an aqueous carrier; and
a pressurized plastic container containing said aqueous composition, wherein said container comprises a hoop tensile strain of about 0.1 to about 1% and wherein said plastic container comprises a gage pressure greater than about 414 kPa.

17. The product of claim 16, wherein said gage pressure is greater than about 690 kPa to about 1000 kPa.

18. The product of claim 16, wherein said hoop tensile strain is about 0.2 to 0.6%.

19. The product of claim 16, wherein said plastic container is pre-treated with water.

20. The product of claim 16, wherein said composition is free of a flavorant.
